(19) United States
(12) Patent Application Publication
(10) Pub. No.: US 2005/0049178 A1
(43) Pub. Date: Mar. 3, 2005

(54) Agent for the occlusion of blood vessels

(75) Inventor: Peter J.W. Sterk, Hamburg (DE)

Correspondence Address: FINNEGAN, HENDERSON, FARABOW, GARRETT & DUNNER;LLP, 1300 I STREET, NW, WASHINGTON, DC, 20005, US

(73) Assignee: Oberschwabenklinik GmbH

(21) Appl. No.: 10/926,307

(22) Filed: Aug. 26, 2004

Related U.S. Application Data
(63) Continuation of application No. 10/019,775, filed on Apr. 12, 2002.

(30) Foreign Application Priority Data
Jul. 6, 1999 (DE) 299 11 689.1

(51) Int. Cl. A61K 38/00

(52) U.S. Cl. 514/2

(57) ABSTRACT

The invention relates to an agent for occluding blood vessels. Said agent contains at least two components, namely an agent for bringing about a vascular occlusion and a physiologically acceptable dye.

The object of the invention is an agent for the occlusion of blood vessels, which significantly improves the success of surgical procedures, especially of surgical procedures for the removal of carcinoma.

In a method for the embolization of blood vessels already known from European patent application 0 797 988, an anti-angiogenic preparation is introduced into a blood vessel that feeds the tumor to treat carcinoma. The blood supply from the diseased tissue to the healthy tissue and vice-versa is interrupted by the “embolization” of the blood vessel.

It has furthermore already been proposed in DE-OS 197 31 741 to use specific conjugates that comprise a compound capable of fluorescence and a carrier to distinguish between healthy and diseased tissue.

Fibrin glues have also proven useful as agents for the occlusion of vessels. However, the use of conventional fibrin glue in oncology surgery has the disadvantage that so far, it has been very difficult or impossible to distinguish between the diseased tissue to be removed and the healthy issue.

The present invention is therefore based on the problem to provide a suitable agent for the occlusion of blood vessels, which allows a safe distinction between healthy and diseased tissue and can therefore be used advantageously during the surgical removal of the diseased tissue.

The object of the invention is attained with an agent for the occlusion of blood. vessels comprising at least two components, i.e., an agent to effect an occlusion of the vessel and a physiologically safe dye. Especially preferred is an agent that comprises a liquid fibrinogen solution to effect the occlusion of the vessel and can be used in cooperation with a liquid thrombin preparation.

The physiologically safe dye is added to one of the two preparations, generally to the thrombin preparation.

The use of the agent in accordance with the invention results in the advantage that it is possible not only to occlude the individual blood vessels, but to stain them as well and thus render the blood—or lymphatic supply visible. The agent can occlude and stain venous as well as arterial blood vessels, and it can also be used in lymphatic vessels.

With the agent in accordance with the invention, it is furthermore possible to visibly separate healthy tissue from diseased tissue. Because each tissue is supplied by a specific artery and vein and by a specific lymph tract, it may be cut off from the blood supply when the appropriate supplying or evacuating supply. tract is embolized. Doing this, it is irrelevant what tissue tract is embolized. It is only important that the blood supply to the diseased tissue is interrupted, which can be achieved with the embolization of the arterial as well as the venous tissue tracts. Thus, during surgical procedures, the use of the agent in accordance with the invention leads to an occlusion of the vessels that supply the operating area.

For the surgeon, the surgical procedure is significantly simplified by the use of the agent in accordance with the invention because he can now readily distinguish between the diseased and the healthy tissue during the surgery, and can retain as much as possible of the healthy tissue when removing the diseased tissue.

Another advantage of the described agent is that it prevents a diffusion of pathogenic bacteria or body cells into the healthy body tissue because of the occlusion of the blood vessels that supply the operating area. Bacteria, viruses, and tumor cells in particular are therefore fixated in the diseased tissue. The same advantages are obtained when the diseased tissue is infested with parasites, in which case the connection between the healthy tissue and the diseased tissue is also interrupted.

The fibrin glue usable in accordance with the invention is preferably comprised of a stabilized, liquid fibrinogen- and a liquid thrombin preparation. One or both of these preparations should contain a physiologically safe dye that clearly stains the embolized blood vessels. Examples of suitable dyes are methylene blue, quinoline yellow, patent blue, tolonium chloride, indocyanine green and foodstuff—as well as fluorescence dyes.

In addition to this, the tissue glue can contain an added preparation containing the blood coagulation factor XIII, and thus be used as a three-component-glue. It is also possible, however, to mix the blood coagulation factor XIII into the fibrinogen preparation from the beginning, thus making it a two-component glue. In the case of a three-component glue, the mixing ratio of the fibrinogen, Factor XIII and thrombin components can be appropriately chosen to obtain good mechanical properties of the glue. Suitable mixing ratios, for example, are 1:1:1 and approximately 2:1:1 to approximately 10:1:1.

The tissue glue used in accordance with the invention contains a chaotropic substance in the fibrinogen preparation. Primarily arginine, guanidine, citrulline, urea or its derivatives or mixtures thereof have been shown to be suitable chaotropic substances. They are generally added to the fibrinogen preparation in quantities of 0.1 to 1.0 mol per liter, preferably in quantities of less than 0.5 mol per liter.

The properties of the aforementioned new tissue glues are furthermore advantageously influenced by the addition of an antifibrinolytic. Aprotinine, ε-amino caproic acid (EACA), p-amino methyl benzoic acid (PAMBA) or one of their physiologically safe salts or derivatives are primarily used as antifibrinolytic.

Furthermore, the fibrinogen preparation can comprise
an inorganic salt or
one or more physiologically safe salts of organic carboxylic acids, especially citric acid or lactic acid, or
one or more amino acids or
a mono- or disaccharide or
a sugar alcohol
   or one of the mixtures thereof as stabilizers.

The Factor VIII-preparation added to the tissue glue to be used in accordance with the invention must also be stabilized if it is not added to already stabilized fibrinogen. In that case, it is advantageous to add a physiologically safe salt of an organic di-, tri- or tetracarboxylic acid, especially citric acid, and, if necessary, other stabilizers and/or buffer substances for the Factor XIII. Other stabilizers may be
a mono- or disaccharide or a sugar alcohol and/or
an amino acid from the group of the glycine, glycylglycine, alanine, cysteine, histidine., glutamine or a physiologically safe salt of the glutamine- or aspartic acid and/or
a reducing or anti-oxidation agent and/or
a surface-active substance.

They are generally added to the Factor XIII-preparation in a quantity of up to 5 percent-by-weight. Tissue glues of this type are described in the German patent applications DE-A-198 53 033 and DE-A-198 61 158.

In addition to the aforementioned tissue glue, it is also possible to use other known agents to effect an occlusion of vessels, such as, for example, histoacryl glues. Said glues are liquid agents based on acrylate, which are suitable to be injected into the blood vessels under high pressure and then evenly distribute in the tissue in the liquid phase and harden there.

The invention is explained in more detail by means of the examples.

Shown are:

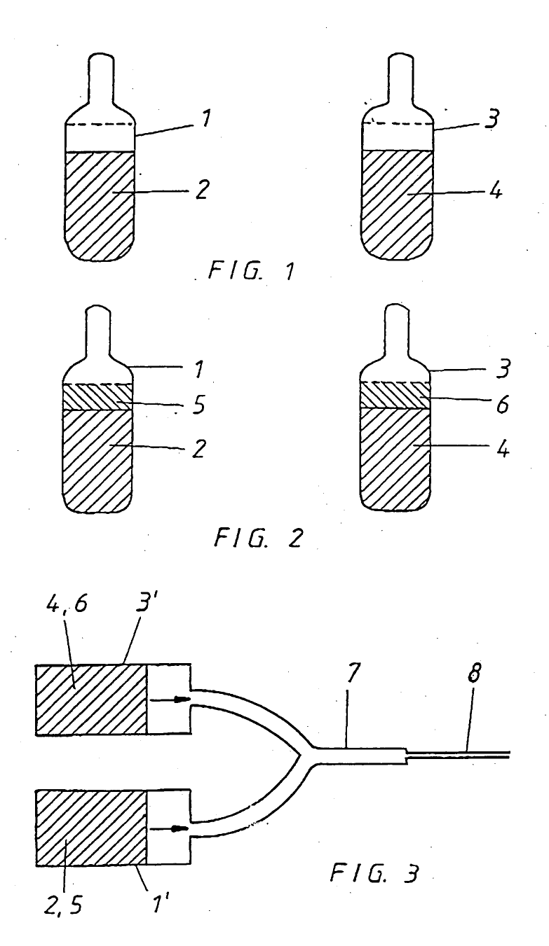

FIG. 1 the representation of two ampoules with various content substances,

FIG. 2 the ampoules in accordance with FIG. 1, with the addition of further additives;

FIG. 3 the application of the agent in a first embodiment;

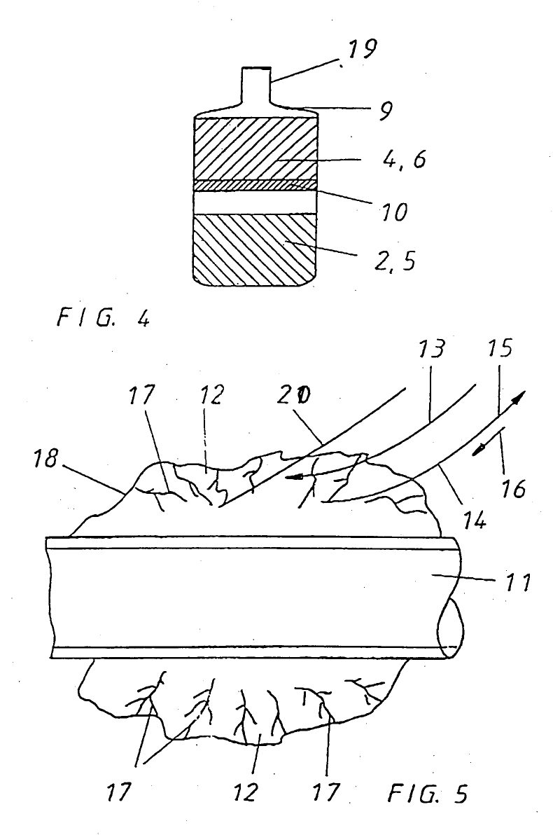

FIG. 4 the application of the agent in a second embodiment, and;

FIG. 5 the explanation of the effect of the agent in the tissue.

FIG. 1 shows two ampoules 1 and 2 that can be designed in various forms. The ampoule 1 can contain a thrombin solution 2, with a physiologically safe dye being dissolved in connection with thrombin in said bottle. The invention is not limited to this; the ampoule 1 may also contain only a dye solution. The thrombin is added only to improve blood coagulation, but is not absolutely necessary for the agent in accordance with the invention.

The ampoule 3 contains a solution of fibrinogen. The fibrinogen is present in a semi-fluid, highly viscous solution.

To prepare the agent in accordance with the invention, an additive 5, which is preferably comprised of a CaCl₂-solution for the later hardening of the agent in the tissue, is placed into the ampoule 1.

An aprotinine solution is placed into the second ampoule 3 as additive 6. A mixing ratio of 1:1 of the aprotinine solution and the fibrinogen solution is preferred.

The additive 6 (aprotinine solution) for the fibrinogen is required to start the desired later coagulation chain.

At first, the content substances of the ampoules 1 and 3 do not react with each other.

A reaction takes place only after, according to FIG. 3, the contents of the two ampoules land 3 are drawn into the assigned syringes 1′ and 3′ and they are connected with one another by a Y-connector according to FIG. 3, as soon as the contents of the two syringes 1′ and 3′ is injected into the tissue through the Y-connector 7 and a cannula 8. FIG. 4 shows as another embodiment a combination vessel 9, which contains the components of the two ampoules 1 and 3 in the embodiment according to FIG. 2.

In the upper part, it can contain the contents of the ampoule 3, while the content substances of the ampoule 1 are in the lower part of the combination vessel 9. The two components are separated by a center membrane 10.

A combination vessel of this type is used in a way that the center, separating membrane 10 is destroyed and the combination vessel is then shaken in such a way that all components are mixed. The agent prepared in this way can then be injected into the issue through the opening 19 and an appropriate cannula 8.

Instead of a horizontal membrane, it is also possible that several horizontal membranes or. one or more vertical membranes may be present in the combination vessel 9.

FIG. 5 shows an example of the application of the agent on a rectum 11. However, the application of the agent is not limited to a rectum; it is also possible to treat living as well as dead tissues in human and animal bodies with the agent in accordance with the invention.

FIG. 5 shows that at position 15, for example, i.e., far away of the diseased tissue, the agent from the cannula 8 is injected into a vein 14 under pressure so that it flows into the direction of the arrow 16 and against the direction of the blood flow in the vein 14.

This stains and simultaneously closes all venous tracts (venules 17) in the affected, diseased tissue 12 and creates the possibility to separate the tissue 12 from the adjacent tissue that is not being supplied by the vein 14. Thus, the adjacent tissue is separated from the diseased tissue 12 by a tissue border 18 and is easily distinguishable. In this way, the diseased tissue 12 can be removed from the adjacent, healthy tissue by a simple, optical control during the surgery.

Another essential advantage of the agent in accordance with the invention is that the diseased tissue has, at least in the border area, closed vessels in which bacteria are immobilized and fixated and thus cannot enter into healthy, not yet diseased tissue.

However, the agent in accordance with the invention can also be injected into an artery 13,.and can then also enter the arterial tracts of the tissue 12 in the direction of the arrow 16, where it closes the arterial tracts there permanently while simultaneously staining them.

It is therefore important for the present invention that the agent is comprised of at least two components, i.e., a substance that is suitable for effecting an embolization of the tissue, and also a dye that is suitable to stain the appropriate occluded tissue during the occlusion.

List of Reference Symbols:

1 Ampoule
2 Filling (colorant solution with or without thrombin)
3 Ampoule
4 Filling (fibrinogen)
5 Additive (CaCl₂)
6 Additive (aprotinine solution)
7 Y-connector
8 Cannula
9 Combination vessel
10 Membrane
11 Rectum
12 Tissue
13 Artery
14 Vein
15 Position
16 Direction of arrow
17 Venules
18 Tissue border
19 Opening
20 Lymph tract

1-4. (canceled)

5. a method for the occlusion of a blood or a lymphatic vessel comprising applying a composition to effect the occlusion of said blood or lymphatic vessel to a desired site,
wherein said composition comprises an agent to effect the occlusion of said blood or lymphatic vessel and a physiologically safe dye,
wherein said composition is suitable for internal application, and
wherein the application of the composition to said desired site stains the occluded blood or lymphatic vessel.

6. A method according to claim 5, wherein the agent to effect the occlusion of said blood or lymphatic vessel comprises a fibrinogen solution.

7. A method according to claim 6, wherein the composition to effect the occlusion of said blood or lymphatic vessel further comprises a thrombin preparation.

8. A method according to claim 7, wherein the composition to effect the occlusion of said blood or lymphatic vessel further comprises blood coagulation factor XIII.

9. A method according to claim 5, wherein the physiologically safe dye is selected from methylene blue, quinoline yellow, patent blue, tolonium chloride, indocyanine green, a foodstuff dye, and a fluorescence dye.

10. A method according to claim 6, wherein the physiologically safe dye is selected from methylene blue, quinoline yellow, patent blue, tolonium chloride, indocyanine green, a foodstuff dye, and a fluorescence dye.

11. A method to distinguish between healthy and diseased tissue comprising
applying a composition to effect the occlusion of a blood or a lymphatic vessel to a desired site, wherein the application of the composition to the desired site stains at least part of the diseased tissue, and
visually distinguishing the stained diseased tissue from the non-stained healthy tissue,
wherein said composition comprises an agent to effect the occlusion of said blood or lymphatic vessel and a physiologically safe dye, and
wherein said composition is suitable for internal application.

12. A method according to claim 11, wherein the agent to effect the occlusion of said blood or lymphatic vessel comprises a fibrinogen solution.

13. A method according to claim 12, wherein the composition to effect the occlusion of said blood or lymphatic vessel further comprises a thrombin preparation.

14. A method according to claim 13, wherein the composition to effect the occlusion of said blood or lymphatic vessel further comprises blood coagulation factor XIII.

15. A method according to claim 11, wherein the physiologically safe dye is selected from methylene blue, quinoline yellow, patent blue, tolonium chloride, indocyanine green, a foodstuff dye, and a fluorescence dye.

16. A method according to claim 12, wherein the physiologically safe dye is selected from methylene blue, quinoline yellow, patent blue, tolonium chloride, indocyanine green, a foodstuff dye, and a fluorescence dye.
